(19) United States
(12) Patent Application Publication
(10) Pub. No.: US 2015/0150624 A1
(43) Pub. Date: Jun. 4, 2015

(54) Device For Therapeutic Delivery of Radio Frequency Energy

(71) Applicant: Jeffrey Petersohn, Haverford, PA (US)

(72) Inventor: Jeffrey Petersohn, Haverford, PA (US)

(21) Appl. No.: 14/481,267

(22) Filed: Sep. 9, 2014

Related U.S. Application Data
(63) Continuation of application No. PCT/US13/30270, filed on Mar. 11, 2013.
(60) Provisional application No. 61/608,881, filed on Mar. 9, 2012.

(51) Int. Cl. A61B 18/14

(52) U.S. Cl. CPC A61B 18/1492; A61B 2017/003

(57) ABSTRACT

An RF catheter includes a catheter shaft extending between a proximal end and a distal end and defining at least one lumen extending therein. An inflatable balloon is attached to the distal end of the catheter shaft and in fluid communication with the at least one lumen for inflation and deflation of the inflatable balloon. At least one electrically conductive element is positioned on at least a portion of an outer surface of the inflatable balloon. The at least one electrically conductive element is electrically connected to at least one electrical connector adjacent the shaft proximal end.

### CROSS-REFERENCE TO RELATED APPLICATIONS

This application is a continuation of PCT Application No. PCT/US13/30270, filed Mar. 11, 2013, which claims the benefit of U.S. Provisional Appln. No. 61/608,881, filed on Mar. 9, 2012, the contents of which are incorporated herein by reference.

### FIELD OF THE INVENTION

The present invention relates to a balloon catheter for delivering radiofrequency energy to a targeted tissue in a patient.

### BACKGROUND OF THE INVENTION

Ablation catheters are well recognized and important tools for conveying an electrical stimulus to selected locations within the human body. Originally, ablation catheters were mainly used for the treatment of certain types of cardiac arrhythmia. For example, such catheters have been used to interrupt or modify existing conduction pathways associated with arrhythmias within the heart. Ablation procedures also are used for the treatment of atrial ventricular (AV) nodal re-entrant tachycardia. Radio frequency (RF) catheter ablation has become increasingly popular for many symptomatic arrhythmias such as AV nodal re-entrant tachycardia, AV reciprocating tachycardia, idiopathic ventricular tachycardia and primary atrial tachycardias. Nath, S. et al., “Basic Aspects Of Radio Frequency Catheter Ablation,” J. CARDIOVASC ELECTROPHYSIOL., Vol. 5, pgs. 863-876, October 1994.

While RF ablation was used initially for cardiac applications, it has also been used for the treatment of chronic pain. Practitioners may also ablate nerve tissue through the use of an electrode surface attached to the end of a catheter shaft. Such RF ablation catheters generally have an arrangement of one or more electrodes at their tip configured to apply RF energy to heat the targeted tissue by resistive heating, creating an ablation lesion that may extend to a depth of several millimeters or more. These catheters may be equipped with coolant supplies to cool the tip and prevent electrode charring.

The standard RF generator used in catheter ablation produces an unmodulated sine wave alternating current at frequencies of approximately 400 to 1000 kHz. The RF energy is typically delivered into the patient between the electrode of the catheter and a large conductive plate in contact with the patient's back. During the delivery of the RF energy, alternating electrical current traverses from the electrode through the intervening tissue to the back plate or ground. The passage of current through the tissue results in electroresistive heating. Heating tissue to temperatures above 50° C. is required to cause irreversible myocardial tissue injury. Heating tissue to temperatures above approximately 100° C. at the electrode tissue interface, however, can result in boiling of plasma and adherence of denatured plasma proteins to the ablation electrode. The formation of this coagulum on the electrode causes a rapid rise in electrical impedance and a fall in thermal conductivity, resulting in a loss of effective tissue heating. Moreover, such extreme heating can damage healthy tissue surrounding the targeted lesion.

In addition to cardiac ablation and the treatment of chronic pain, ablation techniques and RF treatment have been developed for other medical procedures, for example, angiography applications, pulmonary medicine, treatment of airway disorders, orthopedic surgery, gastroenterology and urology.

Such conventional methods and systems generally have been considered satisfactory for their intended purpose. Pinpointing the exact location for tissue ablation, however, can be difficult when ablation electrodes, either implantable or mounted on a catheter, are directed anatomically into the treatment location. Solutions to this problem have been developed to allow for directing electrodes into place visually. Such visual techniques include guiding radio opaque ablation catheters fluoroscopically, or using optic fibers with cameras to visually guide the electrodes into place.

Even visual location techniques can fail to be successful or reliable. Physicians cannot always visually identify the precise location of the tissue which, if ablated, will alleviate the chronic pain. Substantial variation in anatomy is often involved and physicians may need to ablate large amounts of tissue to ensure complete treatment of a small amount of target tissue. As a result, the target nerves are often ablated along with otherwise healthy surrounding tissues. This unnecessary ablation is detrimental because ablation can be permanent and may cause pain due to unnecessary injury to healthy tissue.

Accordingly, there is a need in the art to develop a device that allows for a more precise delivery of RF energy to ablate targeted tissue.

### SUMMARY OF THE INVENTION

In one aspect, the present invention is directed to an RF catheter having a catheter shaft with a hollow shaft having a distal end and a proximal end, the hollow shaft having at least one lumen and at least one electrically conductive wire; and an inflatable balloon attached to the distal end of the flexible member. The inflatable balloon has an outer surface of which at least a portion is electrically conductive and which is connected to the at least one electrically conductive wire. Additionally, at least one lumen allows for inflation and deflation of the inflatable balloon.

The RF catheter may further include an RF generator means connected to the electrically conductive wire to supply RF energy to the electrically conductive portion of the outer surface of the inflatable balloon. Use of the electrical energy to stimulate tissue or measurement of electrical impedance created in application of such energy may be useful in identifying and locating target tissue.

In one embodiment, the inflatable balloon is mounted on the distal end of the catheter shaft in a concentric fashion. In another embodiment, the inflatable balloon is mounted on the distal end of the catheter shaft in an eccentric fashion.

The RF catheter may also include a cooling means for cooling the electrically conductive portion of the outer surface of the inflatable balloon. In this regard, the catheter may also include means for monitoring temperature of the balloon, the catheter and/or surrounding tissue. The temperature monitoring means may be in the form of a thermistor.

In another aspect, the present invention is directed to a method of using the RF catheter described above. This method involves the steps of: (a) inserting the RF catheter in a non-deployed state in a patient; (b) positioning the distal end of the catheter shaft at a desired ablation site; (c) inflating the inflatable balloon until the flexible conductive surface rests against the desired ablation site; (d) providing an effective amount of RF current to the conductive surface to ablate the tissue at the desired ablation site; (e) deflating the inflatable balloon; and (f) withdrawing the ablation catheter from the patient.

### BRIEF DESCRIPTION OF THE FIGURES

FIG. 1 is an overall view of a neural RF balloon catheter system according to one embodiment of the present invention.

FIG. 2 is a cross-sectional view of the distal portion of the embodiment of the neural RF balloon catheter system shown in FIG. 1 in a deployed state.

FIG. 3 is a cross-sectional view of the catheter shaft of the embodiment of the neural RF balloon catheter system shown in FIG. 1.

FIG. 4 is a cross-sectional view of the distal portion of an embodiment of the neural RF balloon catheter system in a deployed state, wherein the balloon is attached to the catheter shaft in a concentric fashion and has a deformable surface.

FIG. 5 is a cross-sectional view of the distal portion of an embodiment of the neural RF balloon catheter system in a deployed state, wherein the balloon is attached to the catheter shaft in an eccentric fashion.

FIG. 6 is a cross-sectional view of the distal portion of another embodiment of the neural RF balloon catheter system in a deployed state, wherein the balloon is attached to the catheter shaft in an eccentric fashion and has a deformable surface.

FIG. 7 is a cross-sectional view of the distal portion of another embodiment of the neural RF balloon catheter system in a deployed state, wherein the balloon is attached to the catheter shaft in a concentric fashion and has a deformable surface upon which an electrode surface sits.

### DETAILED DESCRIPTION

The present invention is directed to a radiofrequency balloon catheter system. This system includes a radiofrequency (RF) signal generator, a catheter shaft, an inflatable balloon and a means for grounding the patient.

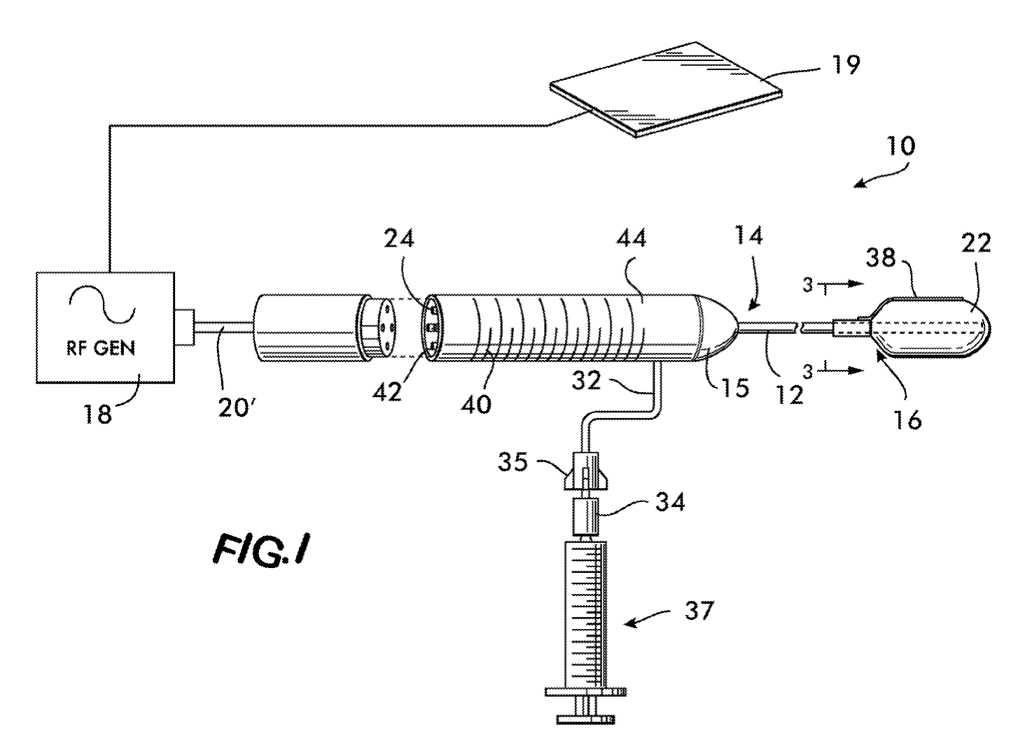

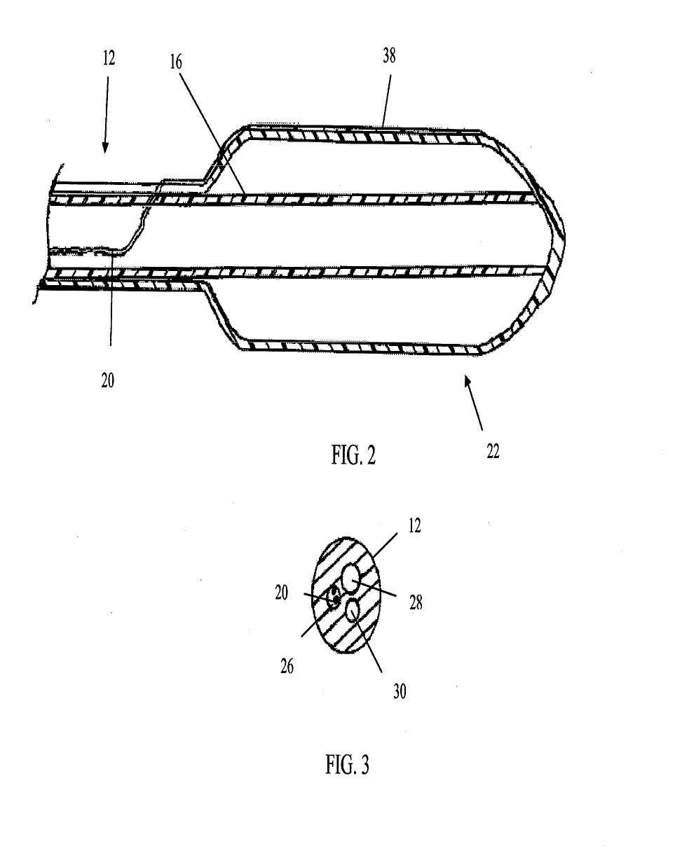

Referring to FIG. 1, a neural RF balloon catheter system 10 is illustrated having a catheter shaft 12 with a distal end 16 and a proximal end 14 extending from a hub 44. An RF generator 18 is attached to the catheter through an electrical conductor, shown in FIG. 1 as electrically conductive wires 20′ which are electrically connected with pins 24 within the hub which in turn are connected to wires 20 extending within the shaft 12 as shown in FIGS. 2 and 3. An inflatable balloon 22 is attached to the distal end 16 of the catheter shaft 12 and is expanded when the catheter is in a deployed state. The balloon 22 may be attached to the catheter shaft 12 by an adhesive such as cyanoacrylate or a medical grade silicone or epoxy adhesive, or similar adhesive that is used in the art. A ground plate or other ground means 19 that contacts a patient is connected to the RF generator 18 may be used to create a closed circuit for the RF current delivery.

The inflatable balloon 22 is constructed of a thin non-latex hypoallergenic material such as polyurethane. The balloon 22 may be attached to the distal end 16 of the catheter shaft 12 or mounted in a concentric or eccentric fashion to the catheter shaft 12. The catheter shaft can be made from any suitable material such as a medical grade stainless steel or a semi-rigid plastic, typically extruded Teflon or polyurethane. The catheter shaft 12 may include a stabilizing braid or strut (not shown) or the like thereabout to add rigidity to the shaft. As shown in FIG. 3, the catheter shaft 12 includes an inflation lumen 28 that extends the length of the catheter shaft 12. This lumen 28 enables the inflation and deflation of the balloon 22 through the injection and withdrawal of air or liquid. When the balloon 22 is inflated, the balloon diameter may be within the range of about 1 mm to about 25 mm, and preferably within the range of about 4 mm to about 8 mm. The diameter may be optimized depending upon the desired application and some embodiments of the balloon may include a non-uniform diameter.

Referring to FIGS. 2 and 3, the catheter shaft 12 may also define a wire lumen 26. One or more electrically conductive wires 20, preferably of stainless steel or copper, are contained within the wire lumen 26. The electrically conductive wires 20 connect the RF generator 18 to one or more electrically conductive segments 38 located on the distal end 16 of the catheter shaft 12 and/or upon the surface of the balloon 22. One or more additional lumens 30 may be defined in the catheter shaft 12 to facilitate passage of a guidewire, for medication delivery, for aspiration of body fluids or tissues as well as for the circulation of coolant. Circulation of a coolant, where used, is typically by means of an external peristaltic roller pump or other pumping mechanism and enables a reduction in the balloon or catheter surface temperature. The coolant, for example, can be water or a physiologic saline solution.

The lumens 26, 28 and 30 may be made by any suitable means, such as molded, extruded, cut or drilled within the catheter shaft 12, or may be formed by use of hollow metal or plastic wires of tubes, which are then incorporated within the catheter shaft 12. In embodiments of the present invention where the catheter shaft is electrically conductive, such as when it is constructed from stainless steel, the catheter shaft is preferably shielded with an insulating material. Alternatively, the catheter shaft may be shielded by an introducing trocar when the Seldinger technique is utilized to position the catheter.

In one embodiment of the present invention, at least one temperature sensing means, such as a thermistor, may be disposed within or on the distal catheter shaft, or within or upon the balloon. Readings from the thermistor are relayed through the use of thermistor connector wires or pins 42 within the hub 44. Use of the temperature sensing means allows the user to carefully control the RF current delivery. A high temperature reading would indicate that the RF current supply should be decreased or shut off, while a low temperature reading indicates a need to increase the RF current supply.

To help the physician effectively place the catheter in the patient, the catheter shaft 12 or hub 44 may be physically marked with various imprinted codes indicating length or depth of catheter insertion, as well as markings 40 that may identify the radial orientation of the catheter, the location of conductive surface(s) of one or more electrodes, and/or the orientation of the balloon 22. The markings 40 may also be used for non-location indicators, such as identification or manufacturing numbers, or date codes.

In certain embodiments of the invention, to further help with the accurate positioning of neural RF balloon catheter system 10, the catheter shaft 12 may incorporate various radio-opaque markings to guide the user of the catheter in properly positioning the device. This ensures that the distal electrode surface is detectable by x-ray fluoroscopy, CT or ultrasound guidance. In other embodiments, microabrasion of the catheter surface will allow visualization by medical ultrasound to guide the operator.

In one embodiment, the neural RF catheter system 10 of the present invention is delivered into the human body by placement inside and advancement through a commercially available styletted introducing stainless steel trocar preferably of about 12-19 gauge. The introducing trocar is typically placed into or adjacent the target tissue structure, such as a nerve, vascular structure or other tissue mass or structure, including an osseous lesion. Typically, a diamond point stylet would be used. In other embodiments, a small wire-wrapped guidewire may be inserted through a commercially available needle or intravenous catheter over which a polyurethane dilator is inserted, removed and replaced by a larger gauge introducing sheath. This is known in the art as the “Seldinger technique”. In another embodiment, the catheter may include a steering wire positioned in or formed within the catheter shaft with a steering portion extending from the proximal end of the catheter. Proximal or distal movement or rotation of the integral steering wire allows controlled directional movement of the catheter.

One or more portions of the distal end of the catheter shaft 16 and/or the surface of the balloon 22 is made electrically conductive by incorporation of an electrically conductive elements 38 on the catheter shaft or by incorporation in or upon the catheter surface and/or the balloon surface of one or more electrically conductive elements 38. The electrically conductive elements 38 are electrically connected to the wires 20. If more than one electrically conductive element 38 is provided, they may all be connected via a singular connection whereby they are all energized together, or they may have individual connections whereby each of the conductive elements 38 can be selectively energized.

The electrically conductive elements 38 may be fabricated from metals, such as a bio-compatible stainless steel, conductive polymers, or other suitable material. The electrically conductive elements 38 may be a wire-like device either extruded, molded, stamped or otherwise formed to conform to the surface of the distal catheter and/or balloon. Other methods for rendering the balloon 22 or catheter surface electrically conductive, including vapor deposition or etching, may be used as well. The electrically conductive element 38 may be a spiral wrapped metal wire, braided metal or other electrically conductive surface, such as stainless steel, although other electrically conductive polymers or other biocompatible materials may be used. This electrically conductive element 38 may be attached to the distal catheter by any suitable means, for example, use of a bio-compatible adhesive such as a medical-grade epoxy, and the element may be attached to one of the electrical conductive wires 20 within the catheter shaft 12 by a variety of commercially available means such as welding or soldering.

The hub 44 may be configured as a steering hub to facilitate guided placement of the shaft distal end 16 through advancing, withdrawing, rotating or otherwise manipulating the catheter device. The hub 44 may further include electrical connectors or pins 24 which are connected via the wire(s) 20 to the electrically conductive elements 38. The wire 20′ of the RF generator 18 has a connector (not shown) configured to connect with the electrical connectors 24. The RF generator 18 preferably produces current in the range of 400-500 kHz, although other suitable ranges may apply.

The proximal end 14 of the catheter shaft 12 also may include connectors, such as the combination of hollow tubing 32 and a Luer-lock connection 34, suitable for the injection, circulation or withdrawal of air or liquid for inflation and deflation of the catheter balloon 22, for example, via an attached syringe 37. A shut-off valve 35 may be provided along the tubing 32. Markings, printed or applied coloration or other means such as molded or applied palpable detents or ridges, can be used to identify the various lumens or connectors.

When the inflating lumen 28 distends the balloon 22, the conductive element 38 or surface on the balloon 22 is moved radially away from the distal end 16 of the catheter shaft 12 to compress tissue against the surface of the balloon 22. Part or all of the conductive balloon 22 and/or catheter surface can be moved against or into proximity of the target tissue structure, deforming the electrically conductive surface 22 against the target tissue or element by means of increased hydraulic pressure within the inflation lumen 28 created by injection of a suitable fluid to distend the balloon 22. The catheter may include a pressure sensor or other pressure monitor, which may be integral with the Luer-lock connection 34, to allow inflation to a desired pressure and to avoid over inflation.

The catheter balloon 22 may be fashioned to produce a variety of shapes upon full inflation depending on the desired application. The balloon 22 and catheter surfaces form one or more electrode equivalents allowing predictable patterns of tissue heating.

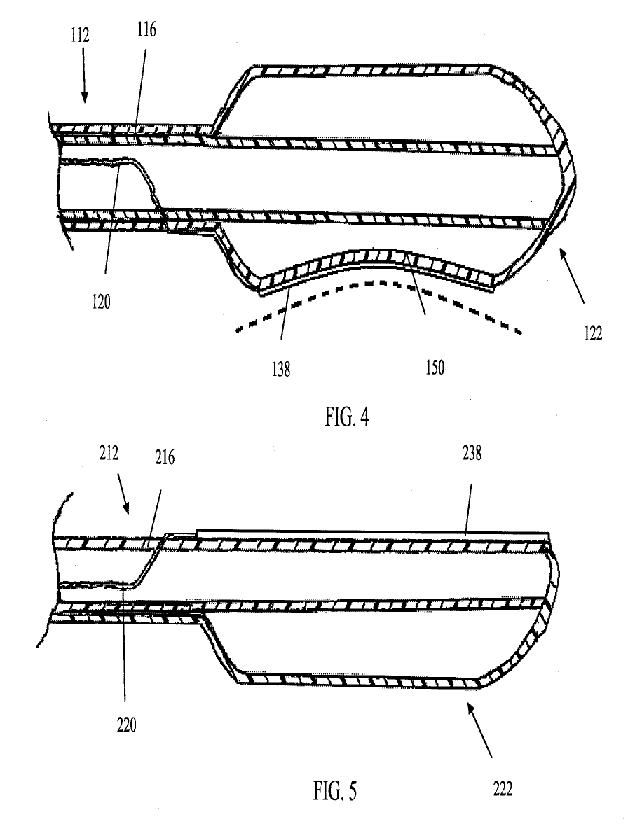

An alternative embodiment of the inflatable catheter balloon is illustrated in FIG. 4. An inflatable balloon 122 is attached in a concentric fashion to a catheter shaft 112 near a distal end 116 of the catheter shaft 112. The inflatable balloon 122 has a deformable surface 150 that deforms when it comes in contact with an internal structure (represented by a broken line). Internal structures that the deformable surface 150 of the balloon 122 may come in contact with will depend on the application, and may include bones, vascular structures, and organs. An electrically conductive segment 138 is attached to the deformable surface 150 to provide an electrode surface that conforms to the adjacent structure. Electrically conductive wires 120 originating from an RF generator are attached to the electrically conductive segment 138 to allow for RF energy to be delivered to the desired location.

FIG. 5 illustrates another embodiment of the present invention. In this embodiment, an inflatable catheter balloon 222 is attached in an eccentric fashion to the catheter shaft 212 on the distal end 216 of the catheter shaft 212. Upon inflation, the balloon 222 will extend away from the catheter shaft 212 in only one direction. An electrically conductive segment 238 is attached to the catheter shaft 212 opposite the balloon 222. Electrically conductive wires 220 originating from an RF generator are attached to the electrically conductive segment 238 to allow for RF energy to be delivered to a desired location.

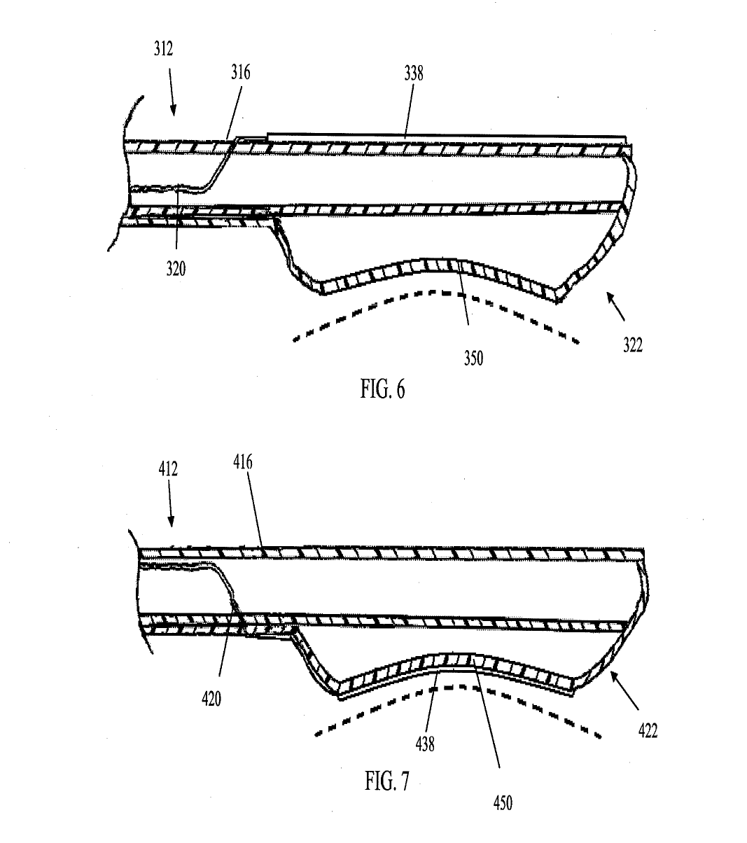

Another embodiment of the present invention is shown in FIG. 6. In this embodiment, an inflatable balloon 322 is attached in an eccentric fashion to a catheter shaft 312 near the distal end of the catheter shaft 316. The balloon 322 has a deformable surface 350 that functions in the same fashion as the embodiment shown in FIG. 4. An electrically conductive segment 338 is attached to the catheter shaft 312 opposite the balloon 322. Electrically conductive wires 320 originating from an RF generator are attached to the electrically conductive segment 338 to allow for RF energy to be delivered to a desired location. In this embodiment, as the balloon 322 inflates, the electrically conductive segment 338 is pushed away f from a surface of an internal structure (represented by a broken line) where heating is to be avoided.

FIG. 7 illustrates an additional embodiment of the present invention. As with the some of the previously described embodiments, an inflatable balloon 422 is attached in an eccentric fashion to a catheter shaft 412 near a distal end of the catheter shaft 416, and as with the embodiment illustrated in FIG. 6, the balloon 422 has a deformable surface 450. This embodiment differs from the embodiment shown in FIG. 6 because an electrically conductive segment 438 is placed on a deformable surface 450 of an inflatable balloon 422. This allows for the delivery of RF energy to a location on or near the surface of an internal structure against which the balloon deforms.

The electrically conductive deformed or inflated balloon and/or catheter surfaces can function as one or more active electrodes in a monopolar or bipolar RF electrical circuit. When RF energy is applied, ionic heating of tissue adjacent to the electrically conductive material results in tissue heating. The tissue heating produces thermocoagulation of protein. Elevated tissue temperature may interrupt neural transmission, cause local interruption of normal blood flow or circulation to a target tissue or may be directly toxic to cells. Use as a bipolar RF electrical circuit allows heating of tissue between two areas, namely between two electrically conductive elements. Additionally, the use of spaced apart conductive elements, either on a single balloon surface or on the balloon surface of two adjacently placed catheters, allows operation without the need for a back or ground plate.

For illustration purposes, provided is a method of inserting an RF balloon catheter system into a patient's body for treating chronic pain, the method comprising: inserting the surface conductive balloon catheter in a non-deployed state at a desired neural ablation site; inflating the balloon until the conductive surface of the balloon rests against the desired neural ablation site; providing RF current to the conductive surface of the inflated balloon to ablate the tissue at the desired site; deflating the balloon; and withdrawing the catheter system from the patient's body.

While use of the invention was described with respect to treating chronic pain, the RF catheter system 10 may be used in various other applications. For example, the system 10 can be used as an angiography catheter, intracranial, extracranial, intracardiac, intrapleural, intravascular, for the therapeutic occlusion of small veins, arteries or arteriovenous malformations, to prevent or stop bleeding from vascular beds, to terminate unwanted arteriovenous, arterioarterial or veno-venous shunts. Similarly, it may be used for the percutaneous treatment of varicose venous with selective thermal ablation or the percutaneous treatment of hypertension by RF thermal lesioning of vascular plexus or sympathetic nerve fibers.

In cardiac applications, the system 10 may be used for therapeutic occlusion of small veins, arteries or arteriovenous malformations, treatment of intracardiac shunts, selective lesion of both intracardiac, intrapericardial or intrapleural vascular or neural structures, and lesions of epicardial tissue. The system helps to minimize damage to the pericardium as well as avoiding adherence of pericardium by displacement of the pericardium from the electrode surface by balloon inflation prior to application of RF energy to the conductive element(s) 38.

In pulmonary medicine, the system 10 may be used for the creation of RF lesions of lung tissue, vagus nerve, or intrathoracic sympathetic nerves with or without thoracoscopic guidance. Inflation of the balloon catheter pushes healthy tissue away from conductive element 38 surfaces and forces the conductive element(s) into anatomic conformity with tissue to be lesioned. When creating RF lesions, this minimizes the formation of unwanted adhesions or scarring between the visceral and parietal pleura.

The system 10 may also be used for the treatment of airway disorders including epistaxis. The balloon 22 is introduced into the naris or oropharynx and directed to target arteriorvenous malformations or sites of bleeding including epistaxis, The balloon 22 is inflated for temporary tamponading of bleeding with subsequent RF heating and thermocoagulation of bleeding blood vessel or arteriovenous malformation. The structure to be heated is thermocoagulated in a distended position, achieving thermocoagulation without tissue shrinkage which would otherwise compromise the luminal diameter. It may also include placement of catheter in the oropharynx, larynx, trachea and bronchial tree for RF thermodestruction of bleeding, malignant or non-malignant lesions. The present visible and steerable RF catheter system 10 causes less tissue destruction than LASER, offers no hazard of fire, smoke nor residua of combustion and can be placed under direct vision or via endoscopy. In an alternative embodiment, the catheter shaft may be sized to fit within an endoscope, urethoscope or the like. In this way, the scope can be guided to a desired tissue location and then the catheter extended therefrom to deliver RF energy in manner described herein.

In gastroenterological applications, the conductive elements 38 may be selectively energized for treatment of bleeding or potential bleeding in a hollow organ or viscus including arteriovenous malformations and sites of bleeding blood vessel or tumor in the esophagus, duodenum, ileum, jejunum, colon, rectum. Target structures may also include or be adjacent to the common bile duct, pancreatic duct or roux-en-y bowel loop. The system may provide minimally invasive delivery of RF energy into post-surgical loop of bowel or hollow viscus or organ for the purpose of tissue lysis including tumor lysis or control of bleeding lesions.

In urological applications, the system 10 allows minimally invasive RF assisted occlusion of aberrant pathway(s) for urine flow including channels of urinary flow development due to malformation, surgery, scarring, infection, deformity or disease resulting in obstruction of normal ureteric system or in the presence of duplicative or aberrant urinary collecting systems. Further applications include RF thermal treatment of bleeding or cancerous lesions of kidney, ureter or bladder and lesioning of sympathetic or other vasomotor nerve structures of, to or from adrenal glands, kidney, ureter or bladder.

In the area of pain treatment, in addition to that described above, the system 10 provides for minimally invasive treatment of intravertebral canal bleeding, cancerous, tumorous or vascular extramedullary lesions of the spinal cord or vertebral canal contents. The system 10 may be further utilized for the production of RF lesions targeting nerves resting upon or adjacent to osseous surfaces. In use, as the balloon 22 is inflated, the conductive element 38 surface(s) is/are pressurized to conform specifically either to or away from osseous surfaces traversed by blood vessels, named or unnamed motor, sensory or sympathetic nerve fibers.

While preferred embodiments of the present invention are described herein, many changes, alterations, modifications and other uses and applications of the present invention will become apparent to those skilled in the art after considering the specification together with the accompanying drawings. All such changes, alterations and modifications that do not depart from the spirit and scope of the invention are deemed to be covered by the invention, which is limited only by the claims that follow.

What is claimed is:

1. An RF catheter comprising:
a catheter shaft extending between a proximal end and a distal end, the shaft defining at least one lumen extending therein;
an inflatable balloon attached to the distal end of the catheter shaft and in fluid communication with the at least one lumen for inflation and deflation of the inflatable balloon, and
at least one electrically conductive element positioned on at least a portion of an outer surface of the inflatable balloon, the at least one electrically conductive element electrically connected to at least one electrical connector adjacent the shaft proximal end.

2. The RF catheter according to claim 1, wherein at least a portion of the distal end of the catheter shaft is electrically conductive.

3. The RF catheter according to claim 1, wherein the inflatable balloon is mounted on the distal end of the catheter shaft in a concentric fashion.

4. The RF catheter according to claim 1, wherein the inflatable balloon is mounted on the distal end of the catheter shaft in an eccentric fashion.

5. The RF catheter according to claim 4, wherein the inflatable balloon has an expandable portion and a non-expandable portion and the at least one electrically conductive element is positioned on the expandable portion.

6. The RF catheter according to claim 4, wherein the inflatable balloon has an expandable portion and a non-expandable portion and the at least one electrically conductive element is positioned on the non-expandable portion.

7. The RF catheter according to claim 1, wherein a portion of the inflatable balloon outer surface defines a deflectable area and the at least on electrically conductive element is positioned on the deflectable area.

8. The RF catheter according to claim 1, wherein a portion of the inflatable balloon outer surface defines a deflectable area and the at least on electrically conductive element is positioned on a portion of the inflatable balloon surface opposite the deflectable area.

9. The RF catheter according to claim 1, wherein the at least one electrically conductive element is fabricated from a metal or a conductive polymer.

10. The RF catheter according to claim 1, wherein the at least one electrically conductive element is in the form of a wire or plate structure secured to the inflatable balloon outer surface.

11. The RF catheter according to claim 1, wherein the at least one electrically conductive element is provided on the inflation balloon outer surface through vapor deposition or etching.

12. The electrically conductive element 38 may be a spiral wrapped metal wire, braided metal or other electrically conductive surface, such as stainless steel, although other electrically conductive polymers or other biocompatible materials may be used.

13. The RF catheter according to claim 1, further comprising a cooling means for cooling the at least one electrically conductive element.

14. The RF catheter according to claim 13, wherein the cooling means includes at least one additional lumen defined in the catheter shaft and configured to supply a coolant to an area proximate the at least one electrically conductive element.

15. An RF catheter system, comprising:
an RF catheter according to claim 1; and
an RF generator connected to the at least one electrical connector, wherein RF current is provided through the electrical connector to the at least one electrically conductive element.

16. The RF catheter system according to claim 15, wherein the RF catheter includes at least two electrically conductive elements and each of the electrically conductive elements is separately electrically connected to the RF generator such that each of the electrically conductive elements may be selectively energized independent of the other electrically conductive elements.

17. A method of using the RF catheter system of claim 15, said method comprising:
inserting the distal end of the catheter shaft into a patient with the inflatable balloon in a non-deployed state;
positioning the distal end of the catheter shaft at a desired treatment site;
aligning the catheter shaft such that the at least one electrically conductive element is positioned as desired relative to the treatment site;
inflating the inflatable balloon to a deployed state;
selectively energizing the at least one electrically conductive element for a desired period;
deflating the inflatable balloon to the non-deployed state; and
withdrawing the RF catheter from the patient.

18. The method according to claim 17 wherein inflation of the inflatable balloon to the deployed state causes the at least one electrically conductive element to be pressed toward the treatment site.

19. The method according to claim 17 wherein inflation of the inflatable balloon to the deployed state causes tissue surrounding the treatment site to be maintained away from at least one electrically conductive element.

20. The method according to claim 17 wherein the RF catheter includes at least two electrically conductive elements and each of the electrically conductive elements is separately electrically connected to the RF generator and the step of selectively energizing the electrically conductive elements includes selectively energizing individual ones of the electrically conductive elements independent of the other electrically conductive elements.
